SEQUENCE LISTING (11 sequences)

SEQ ID NO: 1 - protein, 384 aa, Escherichia coli
MAKHLFTSESVSEGHPDKIADQISDAVLDAILEQDPKARVACETYVKTGMVLVGGEITTSAWVDIEEITRNTVREIGYVHSDMGFDANSCAVLSAIGKQSPDINQGVDRADPLEQGAGDQGLMFGYATNETDVLMPAPITYAHRLVQRQAEVRKNGTLPWLRPDAKSQVTFQYDDGKIVGIDAVVLSTQHSEEIDQKSLQEAVMEEIIKPILPAEWLTSATKFFINPTGRFVIGGPMGDCGLTGRKIIVDTYGGMARHGGGAFSGKDPSKVDRSAAYAARYVAKNIVAAGLADRCEIQVSYAIGVAEPTSIMVETFGTEKVPSEQLTLLVREFFDLRPYGLIQMLDLLHPIYKETAAYGHFGREHFPWEKTDKAQLLRDAAGLK

SEQ ID NO: 2 - DNA, 1155 bp, Escherichia coli
  gene (1)..(1152): metK
atggcaaaacacctttttacgtccgagtccgtctctgaagggcatcctgacaaaattgctgaccaaatttctgatgccgttttagacgcgatcctcgaacaggatccgaaagcacgcgttgcttgcgaaacctacgtaaaaaccggcatggttttagttggcggcgaaatcaccaccagcgcctgggtagacatcgaagagatcacccgtaacaccgttcgcgaaattggctatgtgcattccgacatgggctttgacgctaactcctgtgcggttctgagcgctatcggcaaacagtctcctgacatcaaccagggcgttgaccgtgccgatccgctggaacagggcgcgggtgaccagggtctgatgtttggctacgcaactaatgaaaccgacgtgctgatgccagcacctatcacctatgcacaccgtctggtacagcgtcaggctgaagtgcgtaaaaacggcactctgccgtggctgcgcccggacgcgaaaagccaggtgacttttcagtatgacgacggcaaaatcgttggtatcgatgctgtcgtgctttccactcagcactctgaagagatcgaccagaaatcgctgcaagaagcggtaatggaagagatcatcaagccaattctgcccgctgaatggctgacttctgccaccaaattcttcatcaacccgaccggtcgtttcgttatcggtggcccaatgggtgactgcggtctgactggtcgtaaaattatcgttgatacctacggcggcatggcgcgtcacggtggcggtgcattctctggtaaagatccatcaaaagtggaccgttccgcagcctacgcagcacgttatgtcgcgaaaaacatcgttgctgctggcctggccgatcgttgtgaaattcaggtttcctacgcaatcggcgtggctgaaccgacctccatcatggtagaaactttcggtactgagaaagtgccttctgaacaactgaccctgctggtacgtgagttcttcgacctgcgcccatacggtctgattcagatgctggatctgctgcacccgatctacaaagaaaccgcagcatacggtcactttggtcgtgaacatttcccgtgggaaaaaaccgacaaagcgcagctgctgcgcgatgctgccggtctgaagtaa

SEQ ID NO: 3 - DNA, 29 bp, Artificial Sequence
  Description of Artificial Sequence Oligonucleotide metK2
ccttaattaatgtctgttgtggttggtgt

SEQ ID NO: 4 - DNA, 29 bp, Artificial Sequence
  Description of Artificial Sequence Oligonucleotide metK4
ggaattctctttaggaggtattaaatatg

SEQ ID NO: 5 - DNA, 37 bp, Artificial Sequence
  Description of Artificial Sequence Oligonucleotide RLSS1
ctagcaggaggaattcaccatgggacctgtggatggc

SEQ ID NO: 6 - DNA, 40 bp, Artificial Sequence
  Description of Artificial Sequence Oligonucleotide RLSS2
gggtaccccgctaaaacacaagcttcttggggacctccca

SEQ ID NO: 7 - DNA, 33 bp, Artificial Sequence
  Description of Artificial Sequence Oligonucleotide GAPDHfw
gtcgacgcgtgaggcgagtcagtcgcgtaatgc

SEQ ID NO: 8 - DNA, 43 bp, Artificial Sequence
  Description of Artificial Sequence Oligonucleotide GAPDHrevII
gaccttaattaagatctcatatattccaccagctatttgttag

SEQ ID NO: 9 - DNA, 46 bp, Artificial Sequence
  Description of Artificial Sequence Multiple Cloning Site
gaagatctaggaggcctagcatatgtgaattcccgggctgcagctg

SEQ ID NO: 10 - DNA, 1185 bp, Rattus norvegicus
  CDS (1)..(1185): RLSS-Gen
atgggacctgtggatggcttgtgtgaccattctctaagtgaagagggagccttcatgttcacatctgaatcggtaggagaagggcatccagataagatctgtgaccagattagtgatgcagtgctggatgcccatctcaagcaagaccccaatgccaaggtggcctgtgagacagtgtgcaagacagggatggtgctcctgtgtggagagatcacctcaatggccatgattgactaccagcgggtggtgagagacaccattaagcacattggctacgatgactctgccaagggcttcgacttcaagacctgcaatgtgctcgtggctctggagcaacagtccccagacattgcccaatgtgtccatctagacagaaatgaggaggacgttggtgcaggagatcagggtctgatgttcggctatgccactgacgagacagaggagtgcatgccgctcaccattgttcttgctcacaaactcaacacccggatggcagatctgaggcgctctggtgtccttccctggctgagacctgattctaagactcaggtaacagttcagtacgtgcaggataatggtgctgtcatccctgttcgcgtccacaccatcgtcatctctgtgcaacacaacgaagacataacactggaggccatgcgagaggccctgaaggagcaggtgatcaaagctgtggtgccagccaagtacctggatgaagacaccatctaccacctgcagccaagtgggcggtttgtcatcggaggtccccagggggatgcaggtgtcacaggccgcaagattattgtggacacatacggaggctggggtgcccatggtggtggtgccttctctggaaaggactacaccaaggtggaccgctcagcagcttatgccgcccgctgggtggccaagtctctggtgaaggctgggctctgccggagagtccttgttcaggtgtcctatgccattggtgtggcagaacctctgtccatttccattttcacctacggaacttccaagaagaccgagcgagagctactagaggttgtgaacaagaactttgacctccggccgggtgttattgtcagggacttggatctgaagaagcccatctaccagaagactgcatgctatggtcatttcggaagaagcgagtttccctgggaggtccccaagaagcttgtgttt

SEQ ID NO: 11 - protein, 395 aa, Rattus norvegicus
MGPVDGLCDHSLSEEGAFMFTSESVGEGHPDKICDQISDAVLDAHLKQDPNAKVACETVCKTGMVLLCGEITSMAMIDYQRVVRDTIKHIGYDDSAKGFDFKTCNVLVALEQQSPDIAQCVHLDRNEEDVGAGDQGLMFGYATDETEECMPLTIVLAHKLNTRMADLRRSGVLPWLRPDSKTQVTVQYVQDNGAVIPVRVHTIVISVQHNEDITLEAMREALKEQVIKAVVPAKYLDEDTIYHLQPSGRFVIGGPQGDAGVTGRKIIVDTYGGWGAHGGGAFSGKDYTKVDRSAAYAARWVAKSLVKAGLCRRVLVQVSYAIGVAEPLSISIFTYGTSKKTERELLEVVNKNFDLRPGVIVRDLDLKKPIYQKTACYGHFGRSEFPWEVPKKLVF

(19) United States
(12) Patent Application Publication
(10) Pub. No.: US 2004/0175805 A1
(43) Pub. Date: Sep. 9, 2004

(54) Method for fermentative preparation of S-adenosylmethionine

(75) Inventors: Susanne Leonhartsberger, Munchen (DE); Thomas Maier, Dachau (DE)

Correspondence Address: COLLARD & ROE, P.C., 1077 Northern Boulevard, Roslyn, NY, 11756, US

(73) Assignee: Consortium Fur Electrochemische Industrie GmbH

(21) Appl. No.: 10/789,493

(22) Filed: Feb. 27, 2004

(30) Foreign Application Priority Data
Mar. 6, 2003 (DE) 103 09 856.9

(51) Int. Cl. C12Q 1/68; C12P 19/30; C12N 1/21; C12N 15/74

(52) U.S. Cl. 435/89; 435/252.33; 435/488

(57) ABSTRACT

A method for fermentative production of S-adenosylmethionine (SAM), includes culturing a bacterial strain obtainable from a starting strain and having increased SAM-synthetase activity, compared to the starting strain, in a culture medium, the bacterial strain secreting SAM into the culture medium and the SAM being removed from the culture medium.

### BACKGROUND OF THE INVENTION

1. Field of the Invention

The present invention relates to a method for fermentative preparation of S-adenosylmethionine by using a bacterial strain which overproduces S-adenosylmethionine synthetase.

2. The Prior Art

S-Adenosylmethionine (SAM) is the most important metabolic methyl group donor and is used in the pharmaceuticals sector in the treatment of depressions, diseases of the liver and arthritis. Methods of preparing SAM which have been described comprise growing yeasts (Schlenk F. and DePalma R. E., J. Biol. Chem. 1037-1050 (1957), Shiozaki S. et al., Agric. Biol. Chem. 53, 3269-3274 (1989)) in the presence of the precursor L-methionine and chromatographic purification of the SAM produced, after extraction from the cell lysate (U.S. Pat. No. 4,562,149). A disadvantage of this method is especially the complicated purification of the SAM produced, since the cells have to be disrupted first and SAM has to be removed from all other cell components such as amino acids, sugars, lipids, nucleotides, proteins, cofactors and other high molecular and low molecular weight compounds. For this reason, the development of a method for fermentative production of SAM would have a distinct advantage over current methods, if a selective secretion of the SAM produced into the culture supernatant and thus simplification of the purification method were possible. The culture supernatant contains only a few substances, and secretion of SAM would therefore already be a first purification step and markedly facilitate further purification.

GB1,436,509 describes a method for extracellular production of SAM by yeasts such as Candida tropicalis, for example. A disadvantage of this method is caused by the fact that the producer strains used are unusual fungi which do not have GRAS (generally recognized as safe) status, but are partially even to be classified as pathogenic organisms. Moreover, said organisms are difficult to access by genetic methods and their metabolism is largely unknown. Thus, two substantial requirements for improvement by metabolic engineering are absent. In contrast, bacteria are readily accessible genetically, the metabolism of a plurality of species is well researched and there are many apathogenic species which have GRAS status. A method in which bacteria produce SAM would therefore be very desirable. However, extracellular production of SAM by bacteria is not known yet.

The synthesis of S-adenosylmethionine was studied particularly intensively in the bacterium Escherichia coli (E. coli) (Greene, R. C., Biosynthesis of Methionine in: Neidhardt F. C., Escherichia coli and Salmonella typhimurium, Cellular and Molecular Biology, Second Edition, ASM Press, Washington D.C. (1996), pages 542-560 and references included therein). SAM is synthesized in a single step from L-methionine and ATP, following the complicated and highly regulated synthesis of L-methionine. In the process, all three phosphate groups of ATP are released to give inorganic phosphate and pyrophosphate. This reaction is catalyzed by the enzyme S-adenosylmethionine synthetase (EC 2.5.1.6, methionine adenosyl transferase, SAM synthetase) which is encoded in E. coli by the gene metK. This enzyme has been characterized in detail both biochemically and genetically and exhibits very strong feedback regulation, i.e. the activity of the enzyme is strongly inhibited in the presence of an excess of SAM (Markham et al., J. Biol. Chem., 9082-9092 (1980)). Said feedback regulation prevents an energy-consuming unnecessary synthesis of SAM and cellular SAM levels which are too high and possibly damaging to the cell, but also stands in the way of fermentative overproduction of SAM. SAM synthetases of other organisms (Saccharomyces cerevisiae, Methanococcus janaschii, rats) have also been studied and likewise exhibit an inhibitability by SAM, which is, however, not as pronounced as in SAM synthetase of E. coli (Park et al., Bioorgan. Med. Chem., 2179-2185 (1996); Lu and Markham, J. Biol. Chem., 16624-16631 (2002); Oden and Clarke, Biochemistry, 2978-2986 (1983)).

In contrast to other organisms (e.g. yeast), bacteria do not have an SAM transport system, and bacteria are therefore unable to absorb this substance from the medium, SAM synthetase therefore being an essential enzyme. E. coli SAM synthetase was overproduced, resulting in an increased amount of enzyme in the cell (Markham et al., J. Biol. Chem., 9082-9092 (1980)). However, it is not known whether overproduction also increases the amount of SAM in the cell. This should also not be expected, since accumulation of SAM in the cell is prevented by the abovementioned feedback regulation of SAM synthetase. The regulation of SAM synthetase activity thus limits intracellular production of SAM.

In contrast, overproduction of SAM synthetase from rat liver markedly increased the intracellular SAM level in E. coli (Alvarez et al., Biochem. J., 557-561 (1994); EP0647712A1). This is possible, because, unlike the homologous enzyme of E. coli, this SAM synthetase is not subject to stringent feedback regulation, and bacterial regulation is thus circumvented. Here too, however, no extracellular accumulation of SAM was observed.

### SUMMARY OF THE INVENTION

It is an object of the present invention to provide a method for fermentative production of SAM by means of bacteria, which method markedly simplifies purification of SAM.

The above object is achieved according to the present invention by a method which comprises culturing a bacterial strain which is obtainable from a starting strain having an SAM synthetase and which has increased SAM synthetase activity, compared to said starting strain, in a culture medium, said bacterial strain secreting SAM into said culture medium and said SAM being removed from said culture medium.

In view of the fact that SAM synthetase is, as described above, subject to stringent feedback regulation in bacteria, especially in E. coli, it is surprising that an increase in SAM production can be observed with an increase in activity. In particular, overproduced SAM is wholly unexpectedly secreted into the culture supernatant. There exist, as described above, no examples of bacteria releasing fermentatively produced SAM into the culture supernatant. In particular, there is no known transport system for SAM in bacteria, neither can SAM be absorbed from the medium. Passive diffusion to the outside in the case of a large and also charged molecule such as SAM is extremely unlikely. The extracellular concentration of SAM therefore comes as a complete surprise to a person skilled in the art.

The advantages of the method of the present invention arise from increased SAM production and facilitated work-up from the culture supernatant. This method also enables those SAM syhetases to increase SAM production, which are normally subject to stringent product inhibition which prevents intracellular accumulation of SAM in that the SAM produced is secreted into the culture supernatant and thus no longer inhibits SAM synthetase.

In addition, it was surprisingly found that, in contrast to the prior art, D,L-methionine may also be employed as precursor in the method of the invention, instead of L-methionine. The former is considerably less expensive and thus allows production costs to be drastically reduced.

The present invention thus also relates to a method which comprises culturing a bacterial strain obtainable from a starting strain having an SAM synthetase and which has increased SAM synthetase activity, compared to said starting strain, in a culture medium, said bacterial strain secreting SAM into said culture medium and said SAM being removed from said culture medium which contains D,L-methionine.

Preference is given to using as SAM synthetase in the method of the invention a protein comprising the sequence (SEQ ID NO: 1) or functional variants having a sequence similarity to (SEQ ID NO: 1) of greater than 40%.

The sequence similarity to (SEQ ID NO: 1) is preferably greater than 60%, and particularly preferably greater than 80%.

All the homology values mentioned in the present invention relate to results obtained using the BESTFIT algorithm (GCG Wisconsin Package, Genetics Computer Group (GCG) Madison, Wis.). The invention also relates to the abovementioned SAM synthetases.

Preference is given to using in the method of the invention a gene for one of the abovementioned SAM synthetases, also referred to as metK gene hereinbelow. This is a gene having the sequence (SEQ ID NO: 2) or a functional variant of said gene.

A functional variant means in accordance with the present invention a DNA sequence which is derived from the sequence depicted in (SEQ ID NO: 2) by deletion, insertion or substitution of nucleotides, retaining the enzymic activity of the SAM synthetase encoded by said gene.

An increased activity means in accordance with the present invention preferably that SAM synthetase activity in a bacterial strain used according to the invention has increased by a factor of 2, preferably at least by a factor of 5, compared to the respective starting strain.

Bacterial strains which are used in the method of the invention and which have increased SAM synthetase activity compared to a starting strain may be generated from a starting strain, usually a wild-type strain, using standard molecular-biological techniques.

SAM-synthetase genes were identified in a multiplicity of starting strains. Bacterial strains used in the method of the invention can thus preferably be prepared from starting strains of prokaryotic organisms which are accessible to recombinant methods, are culturable by fermentation and are capable of secreting SAM into the culture medium. They are preferably bacterial strains of the family Enterobacteriaceae, particularly preferably of the species Escherichia, very particularly strains of the species Escherichia coli. Preference is given here in particular to using an E. coli strain which contains no foreign genes.

An increased SAM synthetase activity, compared to starting strains, may in principle be achieved by various approaches.

On the one hand, the gene for SAM synthetase may be modified in such a way that the enzyme encoded thereby has a higher activity than the starting enzyme. This may be effected, for example, by unspecific or specific mutagenesis of an SAM synthetase gene. Unspecific mutations may be produced, for example, using chemical agents (e.g. 1-methyl-3-nitro-1-nitrosoguanidine, ethyl methanesulfonic acid, and the like) and/or physical methods and/or PCR reactions carried out under particular conditions and/or DNA amplification in mutator strains (e.g. XL1-Red, Stratagene, Amsterdam, NL). Methods for introducing mutations at specific positions within a DNA fragment are known. Another possibility of generating SAM synthetases having increased activity, compared to the starting enzyme, is to combine various abovementioned methods.

Another possibility of obtaining increased SAM synthetase activity, compared to starting strains, is to overexpress the gene coding for this enzyme. Overexpression means in accordance with the present invention preferably that the SAM synthetase gene is increasingly expressed by at least a factor of 2, preferably at least a factor of 5, compared to the particular starting strain from which the SAM synthetase gene has been obtained.

A bacterial strain may have an increased copy number of the metK gene in order to achieve overexpression of said metK gene in said strain, and/or expression of the metK gene may be increased, preferably via suitable promoters.

The copy number of a metK gene in a cell of a starting strain may be increased using methods known to the skilled worker. Thus, for example, a metK gene may be cloned into a plasmid vector having multiple copies per cell (e.g. pUC19, pBR322, pACYC184 for Escherichia coli) and introduced into the strain. Alternatively, a metK gene may be integrated several times into the chromosome of a cell. Integration methods which may be utilized are the known systems employing temperate bacteriophages or integrative plasmids or else integration via homologous recombination.

Preference is given to increasing the copy number by cloning a metK gene into a plasmid vector under the control of a promoter. Particular preference is given to increasing the copy number in Escherichia coli by cloning a metK gene into a pBR322 derivative such as, for example, pJF118ut (derived from pJF118EH, Fürste et al. Gene, 119-131 (1986)).

A suitable control region for expressing a plasmid-encoded metK gene is the natural promoter and operator region of said metK gene, but expression of a metK gene may in particular also be increased by means of other promoters. Corresponding promoter systems which make possible either constitutive or controlled, inducible expression of the SAM synthetase gene, such as, for example, the constitutive GAPDH promoter of the gapA gene or the inducible lac, tac, trc, lambda, ara or tet promoters in Escherichia coli, are known to the skilled worker. Such constructs may be used in a manner known per se either on plasmids or chromosomally.

A particularly preferred embodiment of cloning a metK gene makes use of a plasmid which already contains a promoter for increased expression, such as, for example, the inducible tac-promoter system of Escherichia coli.

Furthermore, increased expression may be achieved by translation start signals such as, for example, the ribosomal binding site or start codon of the gene being present in an optimized sequence on the particular construct or by replacing codons which are rare according to “codon usage” with more frequently occurring codons or by optimizing mRNA-stabilizing sequences.

Bacterial strains used in the method of the invention preferably contain a plasmid with a metK gene and the mentioned modifications of the regulatory signals.

A metK gene is cloned into a plasmid vector, for example, by specific amplification of a metK gene by means of the polymerase chain reaction using specific primers which cover the complete metK gene and subsequent ligation with vector DNA fragments.

The efficacy of a bacterial strain for the inventive fermentative production of SAM may be enhanced by additional measures. Instead of adding L-methionine or D,L-methionine, the endogenous methionine synthesis of the strain used in the method of the invention may be strengthened. For this purpose it is possible to use, for example, strains in which the gene metJ which codes for a repressor of the genes of methionine and SAM metabolism is no longer expressed (JP2000139471A) or strains exhibiting improved methionine synthesis, due to their possessing an improved homoserine transsuccinylase (JP2000139471A, DE-A-10247437, DE-A-10249642).

Using a common transformation method (e.g. electroporation, CaCl₂ method) the metK-containing plasmids are introduced into a starting strain and selected for plasmid-carrying clones, for example by means of antibiotic resistance.

The bacterial strain for inventive production of SAM is preferably cultured in a minimal salt medium known from the literature.

Carbon sources which may be used are in principle any utilizable sugars, sugar alcohols, organic acids or salts thereof, starch hydrolyzates, molasses or other substances. Preference is given to using glucose or glycerol. Combined feeding of a plurality of different carbon sources is also possible. Suitable nitrogen sources are urea, ammonia and its salts, nitrate salts and other nitrogen sources. Possible nitrogen sources also include complex amino acid mixtures such as yeast extract, peptone, malt extract, soybean peptone, casamino acids, corn steep liquor and NZ amines.

Furthermore, particular components may be added to the medium, such as vitamins, salts, yeast extract, amino acids and trace elements, which improve cell growth.

Moreover, L-methionine may be added to the medium as specific precursor for SAM synthesis at a concentration of between 0.05 and 25 g/l. Preference is given to adding L-methionine at a concentration of between 1 and 5 g/l.

In a particularly preferred method of the invention, rather than L-methionine, D,L-methionine is added to the medium at a concentration of between 0.05 and 25 g/l. Preference is given to adding D,L-methionine at a concentration of between 1 and 5 g/l.

The strain is preferably incubated under aerobic culturing conditions over a period of 16-150 h and within the range of the optimal growth temperature for the particular strain.

Preference is given to an optimal temperature range of 15-55° C. Particular preference is given to a temperature of between 30 and 37° C.

The strain may be grown in a shaker flask or in a fermentor, with no limitations regarding volume. Culturing may be carried out in a batch process, in a fed-batch process or in a continuous method.

SAM may be obtained from the culture medium according to methods known to the skilled worker, such as centrifugation of the medium to remove the cells and subsequent chromatographic purification, complexing, filtration such as cross flow filtration, for example, or precipitation of the product.

The SAM produced in the method of the invention may be detected and quantified by means of chromatography, for example (e.g. HPLC).

### DETAILED DESCRIPTION OF THE DRAWINGS

Other objects and features of the present invention will become apparent from the following detailed description considered in connection with the accompanying drawings. It should be understood, however, that the drawings are designed for the purpose of illustration only and not as a definition of the limits of the invention.

In the drawing, wherein similar reference characters denote similar elements throughout the several views:

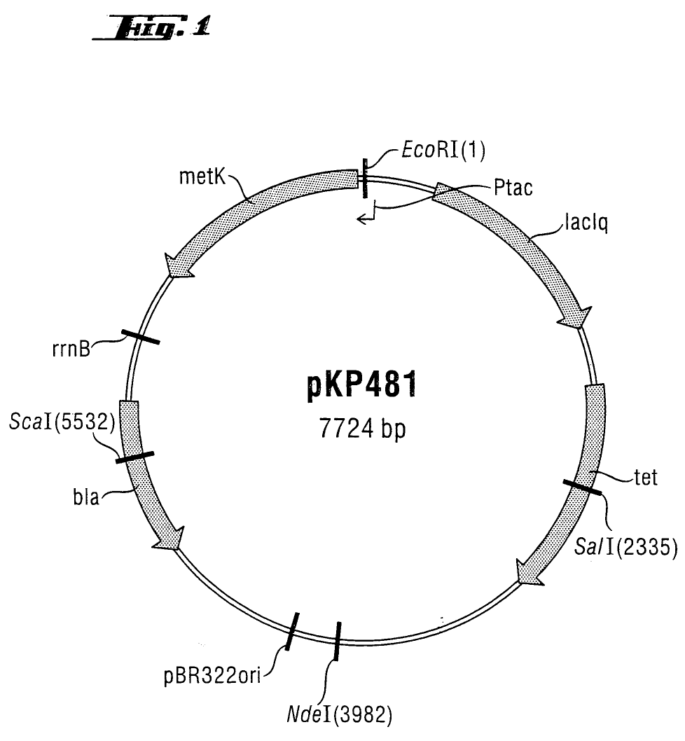

FIG. 1 shows the genetic construction of the plasmid pKP481.

### DETAILED DESCRIPTION OF PREFERRED EMBODIMENT

The following examples serve to further illustrate the invention. The bacterial strain Escherichia coli W3110/pKP481 used for carrying out the examples was deposited with the DSMZ (Deutsche Sammlung für Mikroorganismen und Zellkulturen GmbH, D-38142 Braunschweig) under number DSM 15426, according to the Budapest Treaty.

All molecular-biological methods employed, such as polymerase chain reaction, isolation and purification of DNA, DNA modification by restriction enzymes, Klenow fragment and ligase, transformation etc., were carried out in the manner which is known to a person skilled in the art or is described in the literature or is recommended by the particular manufacturers.

### EXAMPLE 1

### Construction of Plasmid pKP481

A. metK Gene Amplification

The E. coli metk gene was amplified by means of the polymerase chain reaction (PCR) using Taq DNA polymerase according to common practice known to a person skilled in the art. The template used was the chromosomal DNA of E. coli W3110 wild-type strain (ATCC 27325). The primers used were the oligonucleotides metK2, having the sequence 5′-CCTTAATTAATGTCTGTTGTGGTTGGTGT-3′ (SEQ ID No: 3), and metK4, having the sequence 5′-GGAATTCTCTTTAGGAGGTATTAAATATG-3′ (SEQ ID No: 4).

The approx. 1.2 kb DNA fragment obtained in the PCR was then purified by means of a DNA adsorption column of the QIAprep Spin Miniprep Kit (Qiagen, Hilden, Germany), according to the manufacturer's instructions.

B. Cloning of the metK Gene Into the pJF118ut Vector

A cleavage site for EcoRI restriction endonuclease was introduced via primer metK4 into the PCR fragment. The purified PCR fragment was cleaved with EcoRI restriction endonuclease under the conditions indicated by the manufacturer, then phosphorylated, fractionated via an agarose gel and subsequently isolated from said agarose gel by means of the QIAquick gel extraction kit (Qiagen) according to the manufacturer's instructions.

The pJF118ut vector is derived from the cloning and expression vector pJF118EH (Fürste et al. Gene, 119-131 (1986)) and contains various genetic elements which allow controlled expression of any gene. This vector has an origin of replication which is derived from the pBR-plasmid family. Expression of the cloned gene is controlled by the tac promoter, repressed by the lacIq repressor and can be induced by lactose or IPTG.

The metK gene was cloned by cleaving the pJF118ut vector with the EcoRI and PstI restriction enzymes under the conditions indicated by the manufacturer. The 3′ protruding end of the PstI cleavage site was digested by means of Klenow enzyme in the manner known to a person skilled in the art. The 5′ ends of the plasmid were then dephosphorylated by being treated with alkaline phosphatase and subsequently purified, like the PCR fragment, by means of QIAquick gel extraction kit (Qiagen). The PCR fragment was ligated with the cleaved and dephosphorylated vector according to the manufacturer's instructions using T4 DNA ligase. E. coli cells of the DH5a strain were transformed with the ligation mixture by means of electroporation in a manner known to a person skilled in the art. The transformation mixture was applied to LB-ampicillin agar plates (10 g/l tryptone, 5 g/l yeast extract, 5 g/l NaCl, 15 g/l agar, 20 mg/l tetracycline) and incubated at 37° C. overnight.

After plasmid isolation by means of a QIAprep Spin Miniprep Kit (Qiagen, Hilden, Germany), the desired transformants were identified by restriction analysis and their correct sequences confirmed by sequence analysis.

In the plasmid obtained in this way, pKP481 (See FIG. 1), the metK gene is under the control of the tac promoter.

### EXAMPLE 2

### Preparation of an S-adenosylmethionine Producer

The pKP481 plasmid described in Example 1 was transformed into the E. coli strain W3110 (ATCC 27325) by means of the CaCl₂ method and, after selection on LB agar plates containing 20 mg/l tetracycline reisolated from one of the transformants, cleaved with restriction endonucleases and checked. This strain is referred to as W3110/pKP481 and is suitable for SAM production.

### EXAMPLE 3

### Fermentative Production of S-adenosylmethionine

A. Production of SAM

The strain W3110/pKP481 was used for fermentative production of SAM. The W3110 wild-type strain (ATCC 27325), without plasmid and cultured under the same conditions, was used for comparison.

The following medium was used for cultivation: for 1 l of medium: CaCl₂×2H₂O 0.0147 g, MgSO₄×7H₂O 0.3 g, Na₂MoO₄×2H₂O 0.15 mg, H₃BO₃ 2.5 mg, CoCl₂×6H₂O 0.7 mg, CuSO₄×5H₂O 0.25 mg, MnCl₂×4H₂O 1.6 mg, ZnSO₄×7H₂O 0.3 mg, KH₂PO₄ 3.0 g, K₂HPO₄ 12.0 g, (NH₄)₂SO₄ 5 g, NaCl 0.6 g, FeSO₄×7H₂O 0.002 g, Na₃ citrate×2H₂O 1 g, glucose 15 g, tryptone 1 g, yeast extract 0.5 g. For cultivation of W3110/pKP481, 20 μg/ml tetracycline were added to the medium. Where indicated (see table), the medium additionally contained a supplement of 0.5 g/l L-methionine or 1 g/l D,L-methionine.

First, 10 ml of medium in a 100-ml Erlenmeyer flask were inoculated with the appropriate strain and incubated on a shaker at 37° C. and 160 rpm for 16 h to give the preculture for the producer cultivation. The cells prepared in this way were used to finally inoculate 50 ml of the same medium in a 300-ml Erlenmeyer flask to obtain an OD₆₀₀ (absorption at 600 nm) of 0.1. The producer cultures were incubated at 37° C. and 160 rpm on a shaker for 48 h. Expression of the SAM-synthetase gene was induced by adding 0.1 mM isopropyl-β-thiogalactoside (IPTG) at an OD₆₀₀ of 0.6. Samples were taken after 24 h and 48 h, and the cells were removed from the culture medium by centrifugation.

B. Quantification of the SAM Produced

The SAM present in the culture supernatant was quantified by means of HPLC using a Develosil RP-Aqueous C 30 column, 5 mm, 250*4.6 mm (commercially available from Phenomenex, Aschaffenburg, Germany). 10 mL of culture supernatant were applied and fractionated by means of isocratic elution with an eluent of 3 ml of 85% strength H₃PO₄ per 1 l of H₂O at room temperature and a flow rate of 0.5 ml/min and quantified by means of a diode array detector at a wavelength of 260 nm. Table 1 shows the SAM contents obtained in the particular culture supernatant.

TABLE 1

| | S-Adenosylmethionine [mg/l] | | | | | |
|---|---|---|---|---|---|---|
| | Cultivation | | Cultivation with | | Cultivation with | |
| | without | | 0.5 g/l | | 1 g/l | |
| | methionine | | L-methionine | | D,L-methionine | |
| Strain | 24 h | 48 h | 24 h | 48 h | 24 h | 48 h |
| W3110 | 0 | 0 | 0 | 0 | 0 | 0 |
| W3110/pKP481 | 3 | 12 | 61 | 71 | 34 | 31 |

### EXAMPLE 4

### Construction of Plasmid pMSRLSSk

A. RLSS Gene Amplification

The rat liver SAM synthetase (RLSS) gene (Mato et al., Pharmacol. Ther., 265-280 (1997)) was amplified by means of the polymerase chain reaction (PCR) using Taq DNA polymerase according to common practice known to a person skilled in the art. The template used was rat (Rattus norvegicus) cDNA. The primers used were the oligonucleotides RLSS1, having the sequence 5′-CTAGCAGGAGGAATTCACCATGGGACCTGTGGATGGC-3′ (SEQ ID No: 5), and RLSS2, having the sequence 5′-GGGTACCCCGCTAAAACACAAGCTTCTTGGGGACCTCCCA-3′ (SEQ ID No: 6). The approx. 1.2 kb DNA fragment obtained in the PCR was then purified by means of a DNA adsorption column of the QIAprep Spin Miniprep Kit (Qiagen, Hilden, Germany), according to the manufacturer's instructions, and then phosphorylated.

B. Cloning of the RLSS Gene Into Vector

The basic plasmid used for constructing the plasmid of the invention was the pACYC184-derived plasmid pACYC184-LH which has been deposited under number DSM 10172 with the Deutsche Sammlung für Mikroorganismen und Zellkulturen in Braunschweig, Germany. The sequence of the GAPDH promoter was inserted into this plasmid: the GAPDH promoter was amplified by polymerase chain reaction according to the rules known to a person skilled in the art, using the oligonucleotides GAPDHfw, having the sequence 5′-GTCGACGCGTGAGGCGAGTCAGTCGCGTAATGC-3′ (SEQ ID No: 7), and GAPDHrevII, having the sequence 5′-GACCTTAATTAAGATCTCATATATTCCACCAGCTATTTGTTAG-3′ (SEQ ID No: 8), as primers and chromosomal DNA of E. coli W3110 strain (ATCC 27325) as substrate. The product was electrophoretically isolated, purified by means of QIAquick gel extraction kit (Qiagen) and treated with the MluI and PacI restriction enzymes according to the manufacturer's instructions. It was then inserted with the aid of T4 ligase into the pACYC184-LH vector which had been treated with the same enzymes, resulting in plasmid pKP228.

A synthetic multiple cloning site was introduced into the pKP228 plasmid by the following procedure: pKP228 was cleaved with the enzyme BglII, the ends were filled in using Klenow enzyme according to the manufacturer's instructions and dephosphorylated by alkaline phosphatase. A synthesized double-stranded DNA fragment with the following sequence was then inserted into the vector prepared in this way: 5′-GAAGATCTAGGAGGCCTAGCATATGTGAATTCCCGGGCTGCAGCTG-3′ (SEQ ID No: 9). The plasmid produced, pKP504, contains a multiple cloning site downstream of the GAPDH promoter.

pKP504 was cleaved with PvuII, dephosphorylated and ligated according to the manufacturer's instructions and using T4 DNA ligase with the phosphorylated PCR product which contains the gene for rat liver SAM synthetase (gene sequence, see SEQ ID No: 10, protein sequence, see SEQ ID No: 11). E. coli cells of the DH5a strain were transformed with the ligation mixture by means of electroporation in a manner known to the skilled worker. The transformation mixture was applied to LB-ampicillin agar plates (10 g/l tryptone, 5 g/l yeast extract, 5 g/l NaCl, 15 g/l agar, 20 mg/l tetracycline) and incubated at 37° C. overnight.

After plasmid isolation by means of a QIAprep Spin Miniprep Kit (Qiagen, Hilden, Germany), the desired transformants were identified by restriction analysis and their correct sequences confirmed by sequence analysis.

In the plasmid obtained in this way, pMSRLSSk, the RLSS gene (coding for rat liver SAM synthetase) is under the control of the constitutive GAPDH promoter of the Escherichia coli gapA gene.

### EXAMPLE 5

### Preparation of a Second S-adenosylmethionine Producer

The pMSRLSSk plasmid described in Example 4 was transformed by means of the CaCl₂ method into the E. coli W3110 strain (ATCC 27325) and, after selection on LB agar plates containing 20 mg/l tetracycline, reisolated from one of the transformants, cleaved with restriction endonucleases and checked. This strain is referred to as W3110/pMSRLSSk and is suitable for SAM production. The strain was deposited according to the Budapest Treaty with the DSMZ (Deutsche Sammlung für Mikroorganismen und Zellkulturen GmbH, D-38142 Braunschweig, Germany) under number DSM 16133.

### EXAMPLE 6

### Fermentative Production of S-adenosylmethionine

A. Production of SAM

The strain W3110/pMSRLSSk was used for fermentative production of SAM. The W3110 wild-type strain (ATCC 27325), without plasmid and cultured under the same conditions, was used for comparison.

The following medium was used for cultivation: for 1 l of medium: CaCl₂×2H₂O 0.0147 g, MgSO₄×7H₂O 0.3 g, Na₂MoO₄×2H₂O 0.15 mg, H₃BO₃ 2.5 mg, CoCl₂×6H₂O 0.7 mg, CuSO₄×5H₂O 0.25 mg, MnCl₂×4H₂O 1.6 mg, ZnSO₄×7H₂O 0.3 mg, KH₂PO₄ 3.0 g, K₂HPO₄ 12.0 g, (NH₄)₂SO₄ 5 g, NaCl 0.6 g, FeSO₄×7H₂O 0.002 g, Na₃ citrate×2H₂O 1 g, glucose 15 g, tryptone 1 g, yeast extract 0.5 g. For cultivation of W3110/pMSRLSSk 20 μg/ml tetracycline were added to the medium. Where indicated (see table 2), the medium additionally contained a supplement of 0.5 g/l L-methionine or 1 g/l D,L-methionine.

First, 10 ml of medium in a 100-ml Erlenmeyer flask were inoculated with the appropriate strain and incubated on a shaker at 37° C. and 160 rpm for 16 h to give the preculture for the producer cultivation. The cells prepared in this way were used to finally inoculate 50 ml of the same medium in a 300-ml Erlenmeyer flask to obtain an OD₆₀₀ (absorption at 600 nm) of 0.1. The producer cultures were incubated at 37° C. and 160 rpm on a shaker for 48 h. Samples were taken after 24 h and 48 h, and the cells were removed from the culture medium by centrifugation.

B. Quantification of the SAM Produced

The SAM present in the culture supernatant was quantified by means of HPLC using a Develosil RP-Aqueous C 30 column, 5 μm, 250*4.6 mm (commercially available from Phenomenex Aschaffenburg, Germany). 10 μl of culture supernatant were applied and fractionated by means of isocratic elution with an eluent of 3 ml of 85% strength H₃PO₄ per 1 l of H₂O at room temperature and a flow rate of 0.5 ml/min and quantified by means of a diode array detector at a wavelength of 260 nm. Table 2 shows the SAM contents obtained in the particular culture supernatant.

TABLE 2

| | S-Adenosylmethionine [mg/l] | | | | | |
|---|---|---|---|---|---|---|
| | Cultivation | | Cultivation with | | Cultivation with | |
| | without | | 0.5 g/l | | 1 g/l | |
| | methionine | | L-methionine | | D,L-methionine | |
| Strain | 24 h | 48 h | 24 h | 48 h | 24 h | 48 h |
| W3110 | 5 | 0 | 0 | 0 | 0 | 0 |
| W3110/pMSRLSSk | 9 | 46 | 78 | 82 | 63 | 68 |

Accordingly, while a few embodiments of the present invention have been shown and described, it is to be understood that many changes and modifications may be made thereunto without departing from the spirit and scope of the invention as defined in the appended claims.

What is claimed is:

1. A method for fermentative production of S-adenosylmethionine (SAM), which comprises
culturing a bacterial strain obtainable from a starting strain and having increased SAM-synthetase activity, compared to said starting strain, in a culture medium,
said bacterial strain secreting SAM into said culture medium and said SAM being removed from said culture medium.

2. The method as claimed in claim 1,
wherein the bacterial strain used is a strain of the family Enterobacteriaceae.

3. The method as claimed in claim 1,
wherein the bacterial strain used is a strain of the genus Escherichia.

4. The method as claimed in claim 1,
wherein the SAM synthetase used is a protein comprising the sequence (SEQ ID NO: 1).

5. The method as claimed in claim 1,
wherein the SAM synthetase used is a protein comprising a functional variant whose sequence similarity to (SEQ ID NO: 1) is greater than 40%.

6. The method as claimed in claim 1,
wherein the SAM synthetase used is a protein comprising a functional variant whose sequence similarity to (SEQ ID NO: 1) is greater than 60%.

7. The method as claimed in claim 1,
wherein the SAM synthetase used is a protein comprising a functional variant whose sequence similarity to (SEQ ID NO: 1) is greater than 80%.

8. The method as claimed in claim 1, comprising
culturing the bacterial strain in a minimal salt medium.

9. The method as claimed in claim 1,
wherein a carbon source is used and is selected from the groups consisting of glucose and glycerol.

10. The method as claimed in claim 1,
wherein a nitrogen source is used and is selected from the group consisting of urea, ammonia, ammonia salts, and nitrate salts.

11. The method as claimed in claims 1, comprising
incubating the bacterial strain under aerobic culturing conditions over a period of 16-150 h and in the range of the growth temperature optimal for the particular bacterial strain.

12. The method as claimed in claims 1,
wherein L-methionine is added to the minimal salt medium.

13. The method as claimed in claims 1,
wherein L-methionine is added to the minimal salt medium at a concentration of between 0.05 and 25 g/l.

14. The method as claimed in claims 1,
wherein L-methionine is added to the minimal salt medium at concentration of between 1 and 5 g/l.

15. The method as claimed in claim 1,
wherein D,L-methionine is added to the minimal salt medium.

16. The method as claimed in claim 1,
wherein D,L-methionine is added to the minimal salt medium at a concentration of between 0.05 and 25 g/l.

17. The method as claimed in claim 1,
wherein D,L-methionine is added to the minimal salt medium at a concentration of between 1 and 5 g/l.

18. The method as claimed in claims 1,
wherein SAM is recovered from the culture medium by centrifugation of said culture medium and by means selected from the group consisting of subsequent chromatographic purification, complexing, filtration, cross flow filtration, and precipitation of SAM.
